(19) United States
(12) Patent Application Publication
(10) Pub. No.: US 2016/0199147 A1
(43) Pub. Date: Jul. 14, 2016

(54) METHOD AND APPARATUS FOR COORDINATING POSITION OF SURGERY REGION AND SURGICAL TOOL DURING IMAGE GUIDED SURGERY

(71) Applicant: ELECTRONICS AND TELECOMMUNICATIONS RESEARCH INSTITUTE, Daejeon (KR)

(72) Inventor: Ho Chul SHIN, Daejeon (KR)

(21) Appl. No.: 14/991,623

(22) Filed: Jan. 8, 2016

(30) Foreign Application Priority Data
Jan. 12, 2015 (KR) 10-2015-0004208

(51) Int. Cl. A61B 90/00; A61B 1/313; A61B 34/20; A61B 1/00

(52) U.S. Cl. CPC A61B 90/37; A61B 1/00193; A61B 1/3132; A61B 34/20; A61B 2090/374; A61B 2090/3762

(57) ABSTRACT

Provided are a method and an apparatus for providing a guide image for image guided surgery, which is used so as for a doctor to perform accurate surgery, which an determine a position of a surgical tool for a surgery target region of a patient by calculating a position of a laparoscope/endoscope view and the position of the surgical tool for the surgery target region, based on a distance image calculated from a binocular image of a laparoscope/endoscope, prediction of a positional change of a body type/organ by gas injection based on a prior diagnosis image based on CT/MRI, recognition of a posture of the patient and a position of an abdomen through a 3D scanner, recognition of a position/angle of the surgical tool through a surgical tool sensor, and the like.

### CROSS-REFERENCE TO RELATED APPLICATIONS

This application claims priority to and the benefit of Korean Patent Application No. 10-2015-0004208 filed in the Korean Intellectual Property Office on Jan. 12, 2015, the entire contents of which are incorporated herein by reference.

### TECHNICAL FIELD

The present invention relates to a method and an apparatus for providing a guide image for image guided surgery, and particularly, to a method and an apparatus for providing a guide image for image guided surgery, which is used so as for a doctor to perform accurate surgery by providing information on an image, and the like to guide the accurate surgery in the image guided surgery by coordinating positions of a surgery region and a surgical tool.

### BACKGROUND ART

Image guided surgery using a laparoscope or an endoscope as minimum invasion surgery has got a lot of spotlight because a recovery speed of a patient is high and accurate surgery may be performed by optimizing a surgery region. The minimum invasion surgery using image guiding minimizes damage of a human body and increases accuracy and safety of the surgery to increase survival rate and the quality of a life after the surgery.

In the image guided surgery in the related art, medical images of CT, MRI, and the like are used as referential auxiliary images before the surgery and the surgery is performed while verifying a laparoscope or endoscope image during the surgery. However, in the image guided surgery in the related art, it is difficult to determine a position, a sense of distance, and like for a surgery target affected area under the current surgery and a doctor that performs a surgery operation cannot perform the accurate surgery, thereby causing an accident. Accordingly, improvement of a method for providing information of an image for guiding the accurate surgery in the image guided surgery is required.

### SUMMARY OF THE INVENTION

Accordingly, the present invention is contrived to solve the aforementioned problem and the present invention has been made in an effort to provide a method and an apparatus for providing a guide image for image guided surgery, which is used so as for a doctor to perform accurate surgery, which determine a position of a surgical tool for a surgery target region of a patient in real time by calculating a position of a laparoscope/endoscope view and the position of the surgical tool for the surgery target region of the patient, based on a distance image calculated from a binocular image of a laparoscope/endoscope, prediction of a positional change of a body type/organ by gas injection based on a prior diagnosis image based on CT/MRI, recognition of a posture of the patient and a position of an abdomen through a 3D scanner, recognition of a position/angle of the surgical tool through a surgical tool sensor, and the like.

An exemplary embodiment of the present invention provides an apparatus for providing a guide image for image guided surgery through a laparoscope or endoscope, including: an organ shape and position coordination unit coordinating an image to which a transformed shape and a transformed size of an abdomen of a surgery subject analyzed from a CT or MRI based medical image and positional changes of one or more organs are reflected and an image for a posture and an abdomen position of the subject with a 3D distance image for a surgery target region to obtain a final image for a view of the laparoscope or endoscope; a surgical tool position coordination unit coordinating the posture and the abdomen position of the subject with positional information of a surgical tool from a surgical tool sensor to calculate the position of the surgical tool under surgery; and a surgical tool positioning unit deciding the position of an end-portion of the surgical tool for the surgery target region in the final image in real time to display the decided position on a display.

The apparatus may further include: a binocular image input unit inputting a real-time binocular image by photographing the surgery target region by using two or more cameras of the laparoscope or endoscope; a distance image real-time calculation unit generating the 3D distance image from the real-time binocular image; a medical image input unit inputting the CT or MRI based medical image for the subject; and a body type/organic position prediction unit predicting the transformed shape and size of the abdomen of the subject and the positional changes of one or more organs including an organ at the surgery target region by analyzing an abdominal distention degree regarding an obesity index and a body type of the subject due to injection of gas into the subject during laparoscope or endoscope surgery from the medical image.

The apparatus may further include: a 3D scanner generating scanning data regarding an outer region of the abdomen of the subject; and a posture real-time recognition unit generating a real-time 3D contour image for the posture and the abdomen position of the subject from the scanning data.

One or more surgical tool sensors may generate the 3D positional information for real-time recognition of a position to which the surgical tool including the laparoscope, endoscope, or one or more operational tools moves.

Another exemplary embodiment of the present invention provides a method for providing a guide image in an apparatus for providing a guide image for image guided surgery through a laparoscope or endoscope, including: coordinating an image to which a transformed shape and a transformed size of an abdomen of a surgery subject analyzed from a CT or MRI based medical image and positional changes of one or more organs are reflected and an image for a posture and an abdomen position of the subject with a 3D distance image for a surgery target region to obtain a final image for a view of the laparoscope or endoscope; coordinating the posture and the abdomen position of the subject with positional information of a surgical tool from a surgical tool sensor to calculate the position of the surgical tool under surgery; and deciding the position of an end-portion of the surgical tool for the surgery target region in the final image in real time to display the decided position on a display.

The acquiring of the final image may further include inputting a real-time binocular image by photographing the surgery target region by using two or more cameras of the laparoscope or endoscope; generating the 3D distance image from the real-time binocular image; inputting the CT or MRI based medical image for the subject; and predicting the transformed shape and size of the abdomen of the subject and the positional changes of one or more organs including an organ at the surgery target region by analyzing an abdominal distention degree regarding an obesity index and a body type of the subject due to injection of gas into the subject during laparoscope or endoscope surgery from the medical image.

The acquiring of the final image may further include generating scanning data regarding an outer region of the abdomen of the subject; and generating a real-time 3D contour image for the posture and the abdomen position of the subject from the scanning data.

One or more surgical tool sensors may generate the 3D positional information for real-time recognition of a position to which the surgical tool including the laparoscope, endoscope, or one or more operational tools moves.

According to exemplary embodiments of the present invention, a method and an apparatus for providing a guide image for image guided surgery can determine a position of a surgical tool for a surgery target region of a patient in real time by calculating a position of a laparoscope/endoscope view and the position of the surgical tool for the surgery target region of the patient to accurately provide image information of a primary blood vessel or a nerve which is a surgery target region in surgery minimum invasion surgery to a doctor under surgery and primary surgical navigation information for each surgery step, thereby improving surgery success rate.

The exemplary embodiments of the present invention are illustrative only, and various modifications, changes, substitutions, and additions may be made without departing from the technical spirit and scope of the appended claims by those skilled in the art, and it will be appreciated that the modifications and changes are included in the appended claims.

### BRIEF DESCRIPTION OF THE DRAWINGS

FIG. 1 is a diagram for describing an apparatus for providing a guide image for image guided surgery according to an exemplary embodiment of the present invention.

FIG. 2 is a conceptual diagram of positional recognition of a surgery region and a surgical tool in the apparatus for providing a guide image for image guided surgery according to the exemplary embodiment of the present invention.

FIG. 3 is a flowchart for describing a method for providing a guide image in an apparatus for providing a guide image for image guided surgery according to another exemplary embodiment of the present invention.

FIG. 4 is a diagram for describing a hardware implementation example for realizing a function of the method for providing a guide image in the apparatus for providing a guide image for image guided surgery according to the exemplary embodiment of the present invention.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, the present invention will be described in detail with reference to the accompanying drawings. In this case, like reference numerals refer to like elements in the respective drawings. Further, a detailed description of an already known function and/or configuration will be skipped. In contents disclosed hereinbelow, a part required for understanding an operation according to various exemplary embodiments will be described in priority and a description of elements which may obscure the spirit of the present invention will be skipped. Further, some components of the drawings may be enlarged, omitted, or schematically illustrated. An actual size is not fully reflected on the size of each component and therefore, contents disclosed herein are not limited by relative sizes or intervals of the components drawn in the respective drawings.

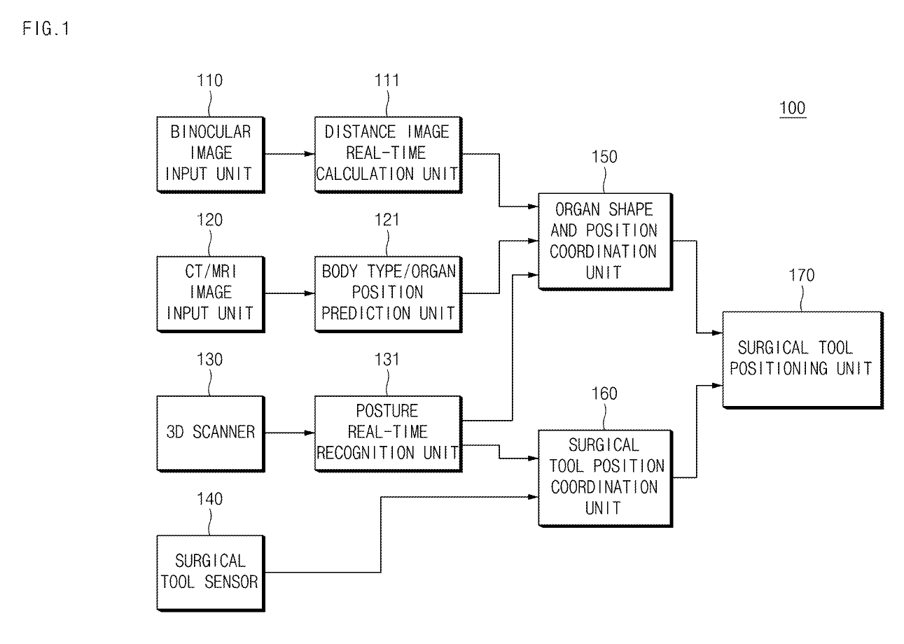

FIG. 1 is a diagram for describing an apparatus 100 for providing a guide image for image guided surgery of a laparoscope or endoscope according to an exemplary embodiment of the present invention.

Referring to FIG. 1, the apparatus 100 for providing a guide image according to the exemplary embodiment of the present invention includes a binocular image input unit 110, a distance image real-time calculation unit 111, a medical image input unit 120, a body type/organ position prediction unit 121, a 3D scanner 130, a posture real-time recognition unit 131, one or more surgical tool sensors 140, an organ shape and position coordination unit 150, a surgical tool position coordination unit 160, and a surgical tool positioning unit 170. The above respective constituent elements of the apparatus 100 for providing a guide image according to the exemplary embodiment of the present invention may be implemented in hardware (e.g., a semiconductor processor, etc.), software, or a combination thereof.

First, functions of the respective constituent elements of the apparatus 100 for providing a guide image according to the exemplary embodiment of the present invention will be described in brief.

The binocular image input unit 110 inputs a real-time binocular image by photographing surgery target regions (organs including a stomach, a heart, a liver, etc.) of surgery subjects including a patient, etc. by using two or more cameras installed in a laparoscope or endoscope.

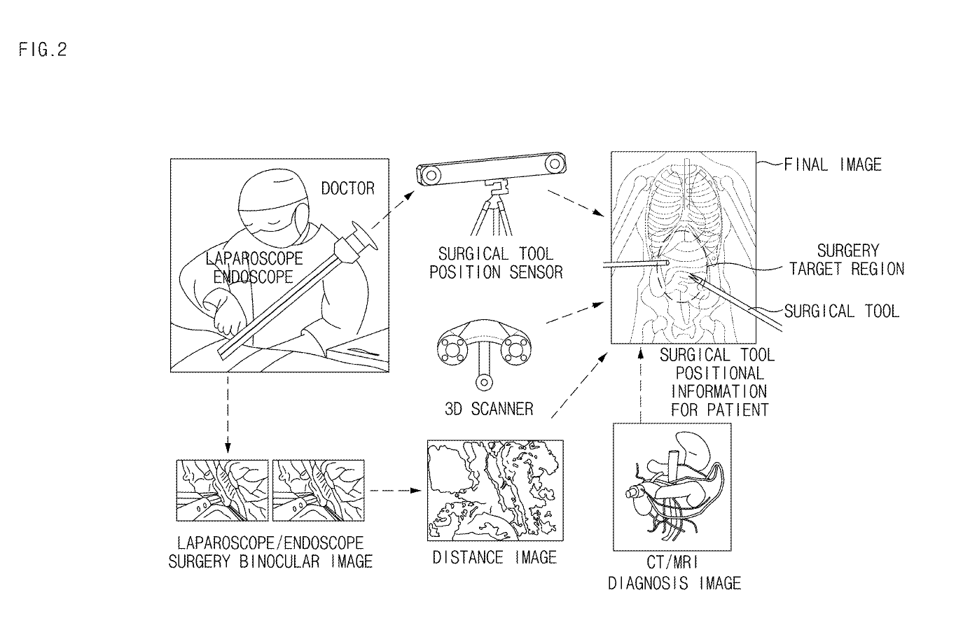

The distance image real-time calculation unit 111 generates a 3D distance image illustrated in FIG. 2 from the real-time binocular image. For example, the distance image real-time calculation unit 111 may generate the 3D distance image so as to display each pixel with a color applied with a distance by using a method that estimates a distance (alternatively, depth) up to a predetermined object included in the binocular image, etc.

The medical image input unit 120 inputs a medical image based on a computed tomography (CT) or magnetic resonance imaging (MRI) apparatus for the surgery subject as illustrated in FIG. 2.

The body type/organ position prediction unit 121 analyzes an abdominal distention degree regarding an obesity index and a body type of the subject due to injection of gas into the subject during the corresponding laparoscope or endoscope surgery from the medical image to predict a transformed shape and size of an abdomen of the subject and positional changes of one or more organs including an organ at a corresponding surgery target region, thereby generating a clear image that enables recognizing the transformed shape and size of the abdomen of the subject and the positions of the organs by reflecting a corresponding prediction result.

The 3D scanner 130 scans an outer region of the abdomen of the subject to generate scanning data. For example, the 3D scanner 130 performs photographing or scanning by using a digital camera, a line camera, and the like to generate the scanning data regarding the outer region of the abdomen of the subject.

The posture real-time recognition unit 131 recognizes a posture and an abdomen position of the subject in real time by appropriately filtering the scanning data (alternatively, image data) of the 3D scanner 130 to generate a clear 3D contour image that enables recognizing the posture and the abdomen position of the subject by reflecting a corresponding recognition result.

The surgical tool sensor 140 generates positional information under surgery for one or more surgical tools including medical procedure tools (e.g., an end effect, etc.) for cutting and suturing of the surgery target region, generating a high frequency, etc., including the laparoscope or endoscope used during surgery using the laparoscope or endoscope to recognize a position to which the corresponding surgical tool moves under the surgery in real time. For example, the surgical tool sensor 140 calculates a relative movement position by analyzing a digital camera image for a motion of the surgical tool to generate 3D positional information. Alternatively, a gyro sensor, an inertial sensor, an acceleration sensor, etc. are mounted on the surgical tool as the surgical tool sensor 140 and the 3D positional information of the surgical tool may be generated by analyzing electrical signals of the sensors.

The organ shape and position coordination unit 150 coordinates an image reflected with the transformed shape and size of the abdomen of the surgery subject and positional changes of one or more organs which the body type/organ position prediction unit 121 analyzes from the medial image based on the CT or MRI and an image for the posture and the abdomen position of the subject recognized by the posture real-time recognition unit 131 with the 3D distance image for the surgery target region from the distance image real-time calculation unit 111 to obtain a final image for a laparoscope or endoscope view as illustrated in FIG. 2.

The surgical tool position coordination unit 160 coordinates the posture and the abdomen position of the subject recognized by the posture real-time recognition unit 131 and the positional information of the surgical tool from the surgical tool sensor 140 to calculate a position (e.g., a 3D coordinate) depending on motion(s) of corresponding surgical tool(s) under the surgery.

The surgical tool positioning unit 170 decides, in real time, positions of end-portions of the surgical tools according to the positions of the surgical tool(s) which the surgical tool position coordination unit 160 calculates with respect to the corresponding surgery target region in the final image from the organ shape and position coordination unit 150 to display the end-portion(s) of the surgical tool(s) in the final image at a corresponding position(s) on a display device (not illustrated) such as an LCD, or the like.

In the present invention, through motions of the respective constituent elements of the apparatus 100 for providing a guide image for image guided surgery according to the exemplary embodiment of the present invention, a position of a surgical tool for a surgery target region of a surgery target patient is determined in real time by calculating a position of a laparoscope/endoscope view and the position of the surgical tool for the surgery target region of the patient to accurately provide image information of a primary blood vessel or a nerve which is a surgery target region in surgery minimum invasion surgery to a doctor under surgery and primary surgical navigation information for each surgery step, thereby improving surgery success rate.

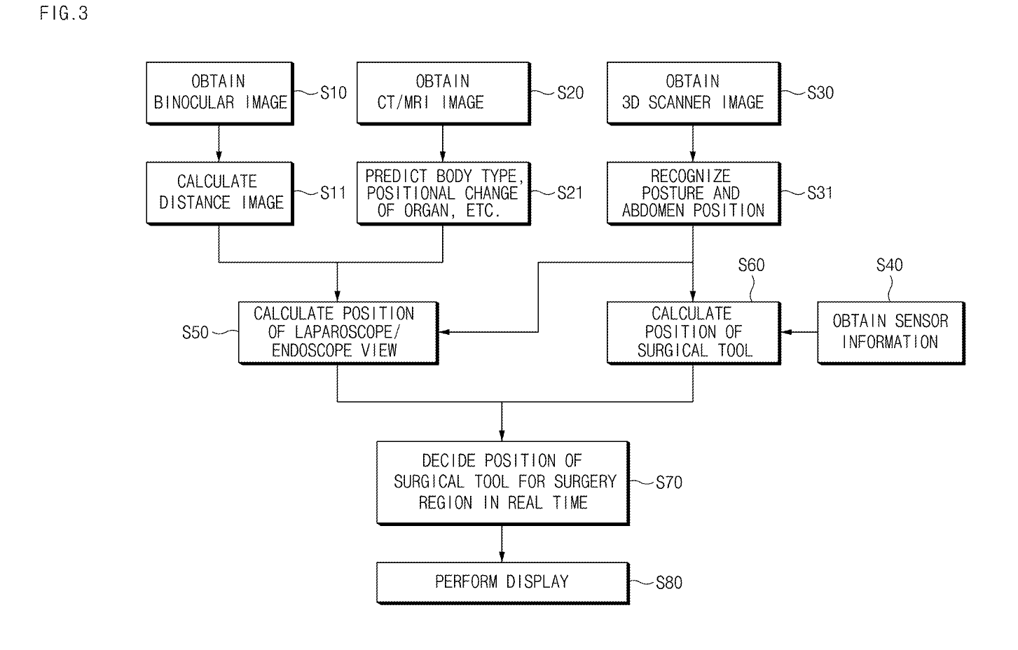

Hereinafter, referring to a flowchart of FIG. 3, a method for providing a guide image in the apparatus 100 for providing a guide image for image guide surgery according to an exemplary embodiment of the present invention will be described in more detail. Description of each step in the method for providing a guide image in the apparatus 100 for providing a guide image for image guide surgery described below is one exemplary and it will be, in advance, revealed that each step needs not particularly performed in order of a symbol of each step and each step may be performed regardless of the order of the symbol if previous data needs not particularly be required in the apparatus 100 for providing a guide image.

It is assumed that in a surgery room having the apparatus 100 for providing a guide image for image guide surgery according to an exemplary embodiment of the present invention, a surgery doctor makes the laparoscope or endoscope, other surgical tools, etc. be close to the surgery target regions (the organs including the stomach, the heart, the liver, etc.) by minimum invasion to perform surgery of the surgery target regions in order to operate the surgery target regions of the surgery target patients.

First, the binocular image input unit 110 may input the real-time binocular image by photographing the surgery target regions (the organs including the stomach, the heart, the liver, etc.) of the surgery subjects by using two or more cameras installed in the laparoscope or endoscope (S10). The distance image real-time calculation unit 111 generates the 3D distance image illustrated in FIG. 2 from the real-time binocular image (S11). For example, similarly to both eyes of a person, according to a predetermined algorithm, the distance image real-time calculation unit 111 may generate the 3D distance image so as to display each pixel with a color applied with a distance by a perspective by using the method that estimates a distance (alternatively, depth) up to a predetermined object included in the binocular image, etc.

The medical image input unit 120 inputs the medical image based on the computed tomography (CT) or magnetic resonance imaging (MRI) apparatus for the surgery subject as illustrated in FIG. 2 (S20). The medical image may be photographed and prepared in advance before the surgery and the medical image input unit 120 may, in advance, input the medical image in the apparatus 100 according to an operation of the apparatus 100 by an operator. The body type/organ position prediction unit 121 analyzes an abdominal distention degree regarding an obesity index and a body type of the subject due to injection of gas into the subject during the corresponding laparoscope or endoscope surgery from the medical image to predict the transformed shape and size of the abdomen of the subject and the positional changes of one or more organs including the organ at the corresponding surgery target region, thereby generating a clear image that enables recognizing the transformed shape and size of the abdomen of the subject and the positions of the organs by reflecting a corresponding prediction result (S21).

Predetermined gas is injected in the abdomen of the surgery subject during the laparoscope or endoscope surgery and in this case, since the abdominal distention degree varies depending on the obesity index and the body type of the subject, the transformed shape and size of the abdomen of the subject and the positions of the organs may be changed. The body type, organic position prediction unit 121 may estimate the obesity index of the subject and the abdomen distension degree depending on the body type by appropriately filtering the medical image by using a predetermined analysis algorithm and predict the transformed shape and size of the abdomen of the subject and the positional change of the organ depending on the abdomen distension degree. The body type, organic position prediction unit 121 may generate a clear image of a surgery target region reflected with the transformed shape and size of the abdomen of the subject and the positional change of the organ depending on the abdomen distension degree and a region therearound.

The 3D scanner 130 scans the outer region of the abdomen of the subject to generate the scanning data (S30). For example, the 3D scanner 130 performs photographing or scanning by using the digital camera, the line camera, and the like to generate the scanning data regarding the outer region of the abdomen of the subject. For example, the scanning data may be generated in order to the 3D contour image by photographing the subject in various directions with a plurality of digital cameras installed around the subject or generate the scanning data for generating the 3D contour image by photographing the subject in various directions while rotating the digital camera, the line camera, etc. around the subject.

The posture real-time recognition unit 131 recognizes the posture and the abdomen position of the subject in real time by appropriately filtering the scanning data (alternatively, image data) of the 3D scanner 130 to generate a clear 3D contour image that enables recognizing the posture and the abdomen position of the subject by reflecting a corresponding recognition result (S31).

As a result, the organ shape and position coordination unit 150 coordinates an image reflected with the transformed shape and size of the abdomen of the surgery subject and the positional changes of one or more organs which the body type/organ position prediction unit 121 analyzes from the medial image based on the CT or MRI and an image for the posture and the abdomen position of the subject recognized by the posture real-time recognition unit 131 with the 3D distance image for the surgery target region from the distance image real-time calculation unit 111 to obtain a final image at a current position view of the laparoscope or endoscope including the image for the surgery target region and the periphery thereof as illustrated in FIG. 2 (S50).

Meanwhile, the surgical tool sensor 140 generates the positional information under surgery for one or more surgical tools including the medical procedure tools (e.g., the end effect, etc.) for cutting and suturing of the surgery target region, generating the high frequency, etc., including the laparoscope or endoscope used during the surgery using the laparoscope or endoscope to recognize a position to which the corresponding surgical tool moves under the surgery in real time (S40). For example, the surgical tool sensor 140 calculates the relative movement position by analyzing the digital camera image for the motion of the surgical tool to generate the 3D positional information. Alternatively, the gyro sensor, the inertial sensor, the acceleration sensor, etc. are mounted on the surgical tool as the surgical tool sensor 140 and the 3D positional information of the surgical tool may be generated by analyzing the electrical signals of the sensors.

The surgical tool position coordination unit 160 coordinates the posture and the abdomen position of the subject recognized by the posture real-time recognition unit 131 and the positional information of the surgical tool from the surgical tool sensor 140 to calculate a position (e.g., a 3D coordinate) depending on motion(s) of corresponding surgical tool(s) under the surgery (S60).

The surgical tool positioning unit 170 decides, in real time, positions of end-portions of the surgical tools in the corresponding image according to the positions of the surgical tool(s) which the surgical tool position coordination unit 160 calculates with respect to the corresponding surgery target region in the final image from the organ shape and position coordination unit 150 (S70) to display the end-portion(s) of the surgical tool(s) in the final image at a corresponding position(s) on a display device (not illustrated) such as an LCD, or the like (S80). The display device may display a 3D coordinate value depending on the motion(s) of the surgical tool(s) as a numerical figure in addition to the image. The image displayed in the display device may be displayed to be expanded or reduced on a screen according to a zoom-in or zoom-out function of a camera of the laparoscope or endoscope.

As described above, through motions of the respective constituent elements of the apparatus 100 for providing a guide image for image guided surgery according to the exemplary embodiment of the present invention, a position of a surgical tool for a surgery target region of a surgery target patient is determined in real time by calculating a position of a laparoscope/endoscope view and the position of the surgical tool for the surgery target region of the patient to accurately provide image information of a primary blood vessel or a nerve which is a surgery target region in surgery minimum invasion surgery to a doctor under surgery and primary surgical navigation information for each surgery step, thereby improving surgery success rate.

The above constituent elements or functions thereof for performing the method for providing a guide image in the apparatus 100 for providing a guide image according to the exemplary embodiment of the present invention may be implemented in hardware, software, or a combination thereof. Furthermore, when the above constituent elements and the functions thereof according to the exemplary embodiment of the present invention are executed by one or more computers or processors, the above constituent elements and the functions thereof may be implemented by a computer or processor-readable code in a computer or processor-readable recording medium. The processor readable recording medium includes every type of recording device in which data readable by a processor is stored. As an example of the processor-readable recording medium, a ROM, a RAM, a CD-ROM, a magnetic tape, a floppy disk, an optical data storage device, and the like are included. Further, the recording medium includes media implemented in a carrier-wave form such as transmission through an Internet. Further, the processor-readable recording media are distributed on computer systems connected through the network, and thus the processor-readable recording media may be stored and executed as the processor-readable code by a distribution scheme.

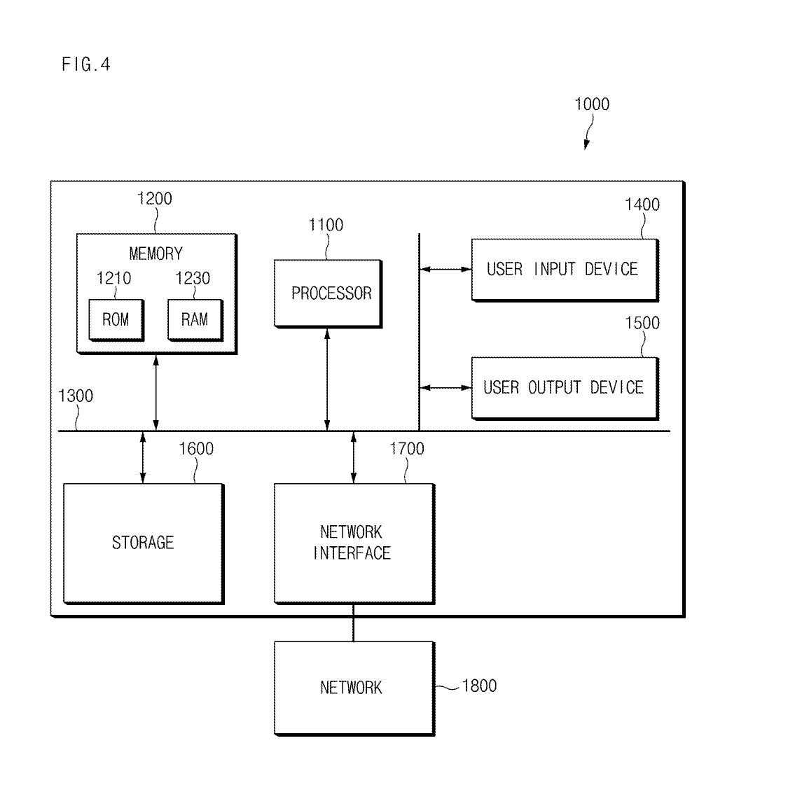

FIG. 4 is a diagram for describing a hardware implementation example for realizing a function of the method for providing a guide image in the apparatus 100 for providing a guide image for image guided surgery according to the exemplary embodiment of the present invention. The constituent elements or the functions for providing a guide image according to the exemplary embodiment of the present invention may be implemented by the hardware, software, or the combination thereof and implemented by the computing system 1000 illustrated in FIG. 4.

The computing system 1000 may include one or more processors 1100 connected through a bus 1200, a memory 1300, a user interface input device 1400, a user interface output device 1500, a storage 1600, and a network interface 1700. The processors 1100 may be a central processing unit (CPU) or a semiconductor device that processes commands stored in the memory 1300 and/or the storage 1600. The memory 1300 and the storage 1600 may include various types of volatile or non-volatile storage media. For example, the memory 1300 may include a read only memory (ROM) and a random access memory (RAM).

Therefore, steps of a method or an algorithm described in association with the exemplary embodiments disclosed in the specification may be directly implemented by hardware and software modules executed by the processor 1100, or a combination thereof The software module may reside in storage media (that is, the memory 1300 and/or the storage 1600) such as a RAM memory, a flash memory, a ROM memory, an EPROM memory, an EEPROM memory, a register, a hard disk, a removable disk, and a CD-ROM. The exemplary storage medium is coupled to the processor 1100 and the processor 1100 may read information from the storage medium and write the information in the storage medium. As another method, the storage medium may be integrated with the processor 1100. The processor and the storage medium may reside in an application specific integrated circuit (ASIC). The ASIC may reside in a user terminal. As yet another method, the processor and the storage medium may reside in the user terminal as individual components.

The specified matters and limited embodiments and drawings such as specific components in the present invention have been disclosed for illustrative purposes, but are not limited thereto, and those skilled in the art will appreciate that various modifications and changes can be made in the art to which the present invention belongs, within the scope without departing from an essential characteristic of the present invention. The spirit of the present invention should not be defined only by the described exemplary embodiments, and it should be appreciated that and claims to be described below and all technical spirits which evenly or equivalently modified are included in the claims of the present invention.

What is claimed is:

1. An apparatus for providing a guide image for image guided surgery through a laparoscope or endoscope, the apparatus comprising:
an organ shape and position coordination unit coordinating an image to which a transformed shape and a transformed size of an abdomen of a surgery subject analyzed from a CT or MRI based medical image and positional changes of one or more organs are reflected and an image for a posture and an abdomen position of the subject with a 3D distance image for a surgery target region to obtain a final image for a view of the laparoscope or endoscope;
a surgical tool position coordination unit coordinating the posture and the abdomen position of the subject with positional information of a surgical tool from a surgical tool sensor to calculate the position of the surgical tool under surgery; and
a surgical tool positioning unit deciding the position of an end-portion of the surgical tool for the surgery target region in the final image in real time to display the decided position on a display.

2. The apparatus of claim 1, further comprising:
a binocular image input unit inputting a real-time binocular image by photographing the surgery target region by using two or more cameras of the laparoscope or endoscope;
a distance image real-time calculation unit generating the 3D distance image from the real-time binocular image;
a medical image input unit inputting the CT or MRI based medical image for the subject; and
a body type/organic position prediction unit predicting the transformed shape and size of the abdomen of the subject and the positional changes of one or more organs including an organ at the surgery target region by analyzing an abdominal distention degree regarding an obesity index and a body type of the subject due to injection of gas into the subject during laparoscope or endoscope surgery from the medical image.

3. The apparatus of claim 1, further comprising:
a 3D scanner generating scanning data regarding an outer region of the abdomen of the subject; and
a posture real-time recognition unit generating a real-time 3D contour image for the posture and the abdomen position of the subject from the scanning data.

4. The apparatus of claim 1, wherein one or more surgical tool sensors generate the 3D positional information for real-time recognition of a position to which the surgical tool including the laparoscope, endoscope, or one or more operational tools moves.

5. A method for providing a guide image in an apparatus for providing a guide image for image guided surgery through a laparoscope or endoscope, the method comprising:
coordinating an image to which a transformed shape and a transformed size of an abdomen of a surgery subject analyzed from a CT or MRI based medical image and positional changes of one or more organs are reflected and an image for a posture and an abdomen position of the subject with a 3D distance image for a surgery target region to obtain a final image for a view of the laparoscope or endoscope;
coordinating the posture and the abdomen position of the subject with positional information of a surgical tool from a surgical tool sensor to calculate the position of the surgical tool under surgery; and
deciding the position of an end-portion of the surgical tool for the surgery target region in the final image in real time to display the decided position on a display.

6. The method of claim 5, wherein the acquiring of the final image includes:
inputting a real-time binocular image by photographing the surgery target region by using two or more cameras of the laparoscope or endoscope;
generating the 3D distance image from the real-time binocular image;
inputting the CT or MRI based medical image for the subject; and
predicting the transformed shape and size of the abdomen of the subject and the positional changes of one or more organs including an organ at the surgery target region by analyzing an abdominal distention degree regarding an obesity index and a body type of the subject due to injection of gas into the subject during laparoscope or endoscope surgery from the medical image.

7. The method of claim 5, wherein the acquiring of the final image includes:
generating scanning data regarding an outer region of the abdomen of the subject; and
generating a real-time 3D contour image for the posture and the abdomen position of the subject from the scanning data.

8. The method of claim 5, wherein one or more surgical tool sensors generate the 3D positional information for real-time recognition of a position to which the surgical tool including the laparoscope, endoscope, or one or more operational tools moves.
